(19) United States
(12) Patent Application Publication
(10) Pub. No.: US 2017/0340689 A1
(43) Pub. Date: Nov. 30, 2017

(54) TITRATED EXTRACTS OF CYNARA SCOLYMUS FOR USE IN THE TREATMENT OF MESOTHELIOMA

(71) Applicant: ABOCA S.P.A SOCIETÀ AGRICOLA, Sansepolcro, AR (IT)

(72) Inventor: Valentino MERCATI, Sansepolcro (IT)

(21) Appl. No.: 15/528,880

(22) PCT Filed: Nov. 24, 2015
(86) PCT No.: PCT/IB2015/059075

§ 371 Date: May 23, 2017

(30) Foreign Application Priority Data
Nov. 25, 2014 (IT) 102014902312096

(51) Int. Cl. A61K 36/28; A61K 31/216; A61K 31/365; A61K 31/519

(52) U.S. Cl. CPC A61K 36/28; A61K 31/519; A61K 31/216; A61K 31/365

(57) ABSTRACT

The present invention relates to a titrated extract of Cynara scolymus, to titrated fractions of extract of Cynara scolymus or titrated mixtures of said extract with one or more of said titrated fractions or mixtures of said fractions and to compositions and kits that comprise them for the prevention and/or the treatment of malignant pleural mesothelioma.

### TECHNICAL FIELD OF THE INVENTION

The present invention relates to a titrated extract of Cynara scolymus, to titrated fractions of extract of Cynara scolymus or titrated mixtures of said extract with one or more of said titrated fractions or mixtures of said fractions and to compositions and kits that comprise them for the prevention and/or the treatment of malignant pleural mesothelioma (MPM).

### PRIOR ART

Malignant pleural mesothelioma (MPM) is an aggressive tumour derived from the mesothelial cells of the chest cavities, and, although chemotherapy (often if pemetrexed is used) improves the survival time in patients with non-operable MPM, the average global survival time is very short. It has been reported recently that a potential molecular therapeutic target for MPM is the interleukine-6 signalling pathway (IL-6)/JAK/STAT3 activated by the high level of IL-6 present in pleural liquid of patients with MPM. The bind of IL-6 to its receptor causes a conformational change in the receptor that initiates JAK activation, which in turn initiates the dimerization of the STAT3 transcription factor, and the STAT3 dimer translocates in the nucleus, thus determining the initiation of the transactivation of various target genes.

This pathway is key for the occurrence of haematopoiesis, of the immune response and of oncogenesis. In addition, it has also been demonstrated that the dysfunction of the JAK/STAT3 system is involved in the development of cancer.

It has been reported in the literature that the exposure to asbestos is one of the causes of mesothelioma.

It is also expected that the incidence of mesothelioma in developed countries will rise in the next 15 years, and some projections have predicted a constant doubling of the cases of hMPM each year since 1998 up to 2018. A dramatic rise of mesothelioma in the third world is also expected, in particular in India, where the use of asbestos continues to rise exponentially without the necessary precautions being taken. Currently, the cases of mesothelioma cause approximately 3,000 deaths per year in the USA and approximately 5,000 in Europe. In spite of the programs of asbestos elimination, the frequencies of mesothelioma have not changed significantly in the last 20 years and it is estimated that they will rise by 5%-10% per year in European countries in the next 25 years.

The WHO estimates that approximately 125 million people are exposed to asbestos in the workplace.

In the United States, the total predicted number of cases of mesothelioma in the male population over the course of 50 years is estimated to be equal to 71,000.

In Europe, where the commercial use of asbestos has been banned for years, a first analysis has predicted that the deaths caused by mesothelioma in the male population will continue to rise with a maximum peak around 2020, or, in accordance with more recent predictions, around 2015 (considering an average latency of 44.6 years).

The annual incidence of the disease varies according to country, however it is suspected that in the emerging markets the number of cases will rise dramatically due to the lack of regulation in asbestos mines and the widespread diffusion of the use of asbestos at industrial and domestic level.

Human MPM (hMPM) is typically classified into 4 sub-groups, of which various prognostic factors have been identified. Current therapies include surgery, radiation, chemotherapy and multimodal therapy, but until now have brought rather disappointing results. Mesothelioma is rarely suitable for radical surgical resection and its resistance to chemotherapy and to radiotherapy is commonly reported in the literature. Average survival from the moment of diagnosis is 8-18 months.

There are still no effective drugs for mesothelioma treatment.

### SUMMARY OF THE INVENTION

The authors of the present invention have demonstrated, as will be seen in the experimental part of the application, in numerous experiments and on various cell lines of malignant pleural mesothelioma, that the extracts described here act in a differential manner on malignant pleural mesothelioma cells (inhibiting their growth by various mechanisms) and on untransformed mesothelial cells.

The authors of the present invention have also characterized the extract, titrating it for some components, and have then isolated different fractions of extract of Cynara scolymus and titrated them for the same components in order to be able to identify, on the one hand, individual fractions with titrations similar to those of the extract of Cynara scolymus used in the reported experiments, and also so as to be able to mix different fractions among them or with said extract so as to obtain an end compound with titrations similar to those of the extract reported in the examples and in the figures, in order to be able to provide standardized preparations suitable for a clinical use.

The inventors of the present disclosure have demonstrated that an extract of Cynara scolymus, titrated as described in the present patent application, is able to inhibit the constitutive or anomalous activation of STAT3 and to induce the reactivation of apoptosis in cultures of MPM tumour cells. In addition, the authors of the present invention have also demonstrated that, in experiments on cultures of MPM tumour cells, the extract of Cynara scolymus inhibits wound healing, in fact preventing the invasivity of the tumour cells. In addition, the authors of the present invention have also demonstrated with experiments of engraftment of tumour cells in mice that the extract of the present invention exerts in vivo an anti-tumour effect with respect to MPM cells.

A first subject of the present invention is therefore an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form

for use in the prevention and/or in the treatment of malignant pleural mesothelioma.

A second subject of the present invention is an extract of Cynara scolymus or a fraction of extract of Cynara scolymus or a mixture of said extract with one or more of said fractions or the mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form for use in the prevention and/or in the treatment of malignant pleural mesothelioma.

A third subject of the present disclosure is a composition comprising, as sole active pharmaceutical ingredient, an extract of Cynara scolymus or a fraction of extract of Cynara scolymus or a mixture of said extract with one or more of said fractions or a mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form and a carrier and/or diluent and/or excipient for use in the prevention and/or in the treatment of malignant pleural mesothelioma.

A fourth subject of the present invention is a composition comprising or consisting in an extract of Cynara scolymus or a fraction of extract of Cynara scolymus or a mixture of said extract with one or more of said fractions or a mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form in association with one or more agents with anti-tumour activity and a carrier and/or diluent and/or excipient for use in the prevention and/or in the treatment of malignant pleural mesothelioma.

A fifth subject of the present invention is a kit for concomitant or sequential administration in association of an extract of Cynara scolymus or a fraction of extract of Cynara scolymus or a mixture of said extract with one or more of said fractions or a mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form and of one or more compounds with anti-inflammatory and/or anti-tumour activity, comprising one or more aliquots of an extract of Cynara scolymus or a fraction of extract of Cynara scolymus or a mixture of said extract with one or more of said fractions or a mixture of said fractions as above defined or one or more aliquots of a composition comprising, as active pharmaceutical ingredient, an extract of Cynara scolymus or a fraction of extract of Cynara scolymus or a mixture of said extract with one or more of said fractions or a mixture of said fractions as defined above and one or more separate aliquots of a chemotherapeutic agent or a mixture of chemotherapeutic agents with suitable pharmaceutically acceptable carriers for use in the prevention and/or in the treatment of malignant pleural mesothelioma.

A sixth subject of the invention is a therapeutic method for the prevention and/or in the treatment of malignant pleural mesothelioma comprising the step of administering to an individual who needs it a therapeutically active quantity of extract of Cynara scolymus or a fraction of extract of Cynara scolymus or a mixture of said extract with one or more of said fractions or a mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form, or of a pharmaceutical composition as above defined, optionally in association with one or more agents having anti-tumour and/or anti-inflammatory activity.

All the subjects described concern malignant pleural mesothelioma.

For the purposes of the present description, the term Cynara scolymus corresponds to the term Cynara cardunculus subsp. scolymus and can be substituted therewith in any point of the description and of the claims.

For the purposes of the present description, the term “comprising” can be substituted in any point of the description and of the claims with the term “consisting of”.

### DETAILED DESCRIPTION OF THE FIGURES

Note: In the present figures, the extract of Cynara spp. used is often indicated by the abbreviation ABO-1.

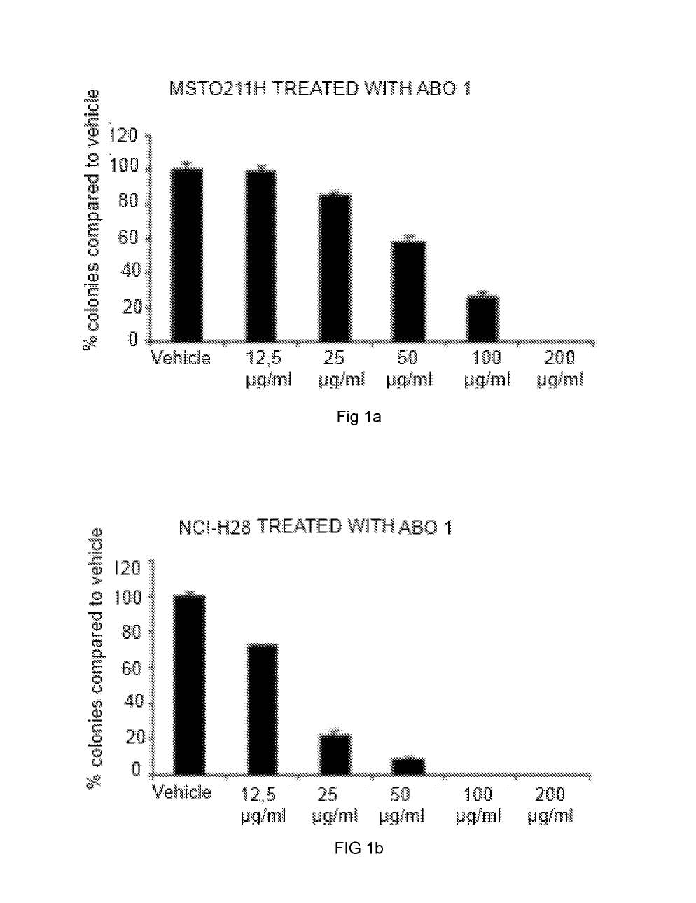

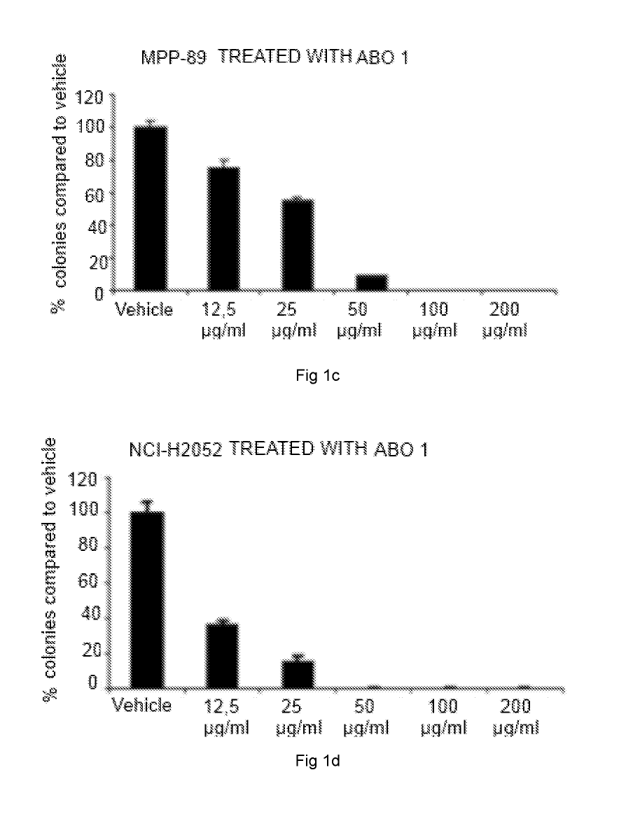

FIG. 1: Clonogenic assay (see the experimental section for the conditions) on cell lines of human malignant pleural mesothelioma with various doses of extract of Cynara scolymus

graph 1a. assay performed on human mesothelioma cell line MSTO211H

graph 1b. assay performed on human mesothelioma cell line NCI-H28

graph 1c. assay performed on human mesothelioma cell line MPP-89

graph 1d. assay performed on human mesothelioma cell line NCI-H2052

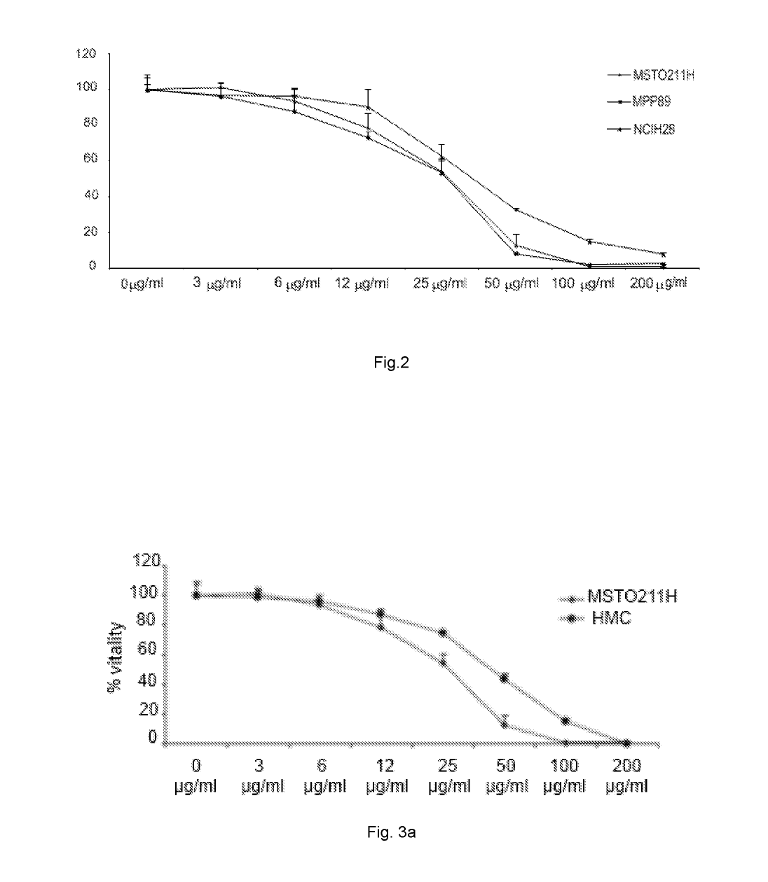

FIG. 2: Assay of cell vitality using ATPlite assay (see the experimental section for the conditions) on malignant pleural mesothelioma cell lines (MSTO211H, MPP-89, NCI-H28). The assay shows that cell vitality is inhibited by the extract of Cynara scolymus of the invention in a dose-dependent manner in various mesothelioma cell lines.

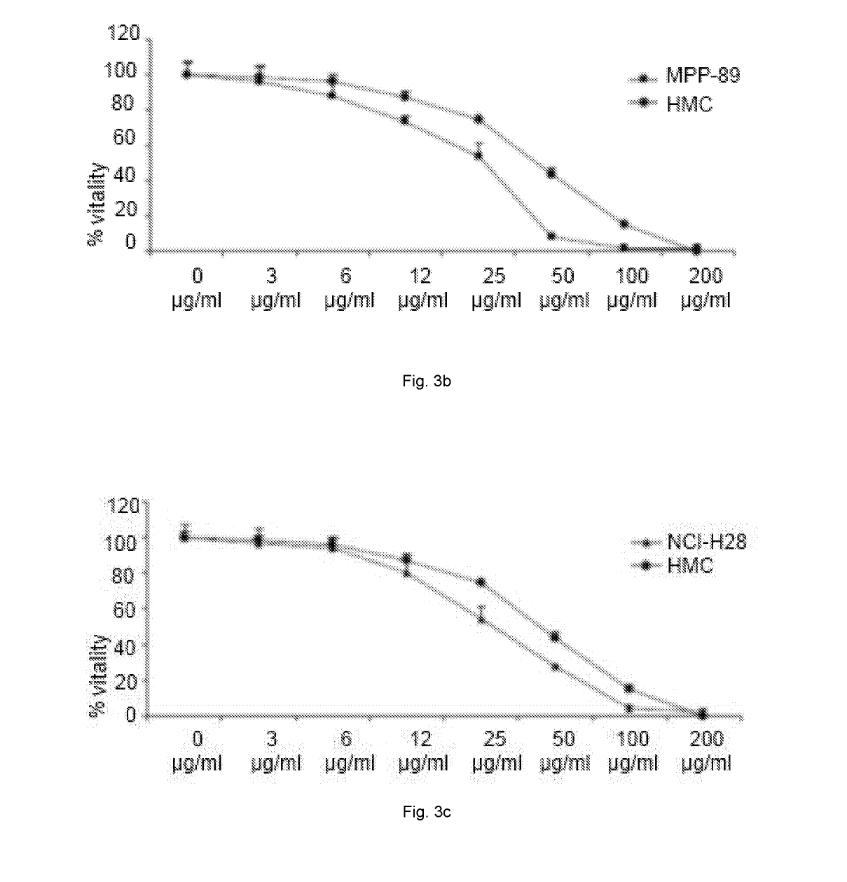

FIG. 3: comparison of the three vitality curves of FIG. 2 compared with (FIG. 3a MSTO211H, FIG. 3b MMP-89, FIG. 3c NCI-H28) the proliferation curve obtained treating normal mesothelial cells (HMC) with extract of Cynara scolymus. The malignant mesothelioma cell lines (MPMs) clearly show the anti-proliferative effect of the extract of Cynara scolymus compared with the HMCs.

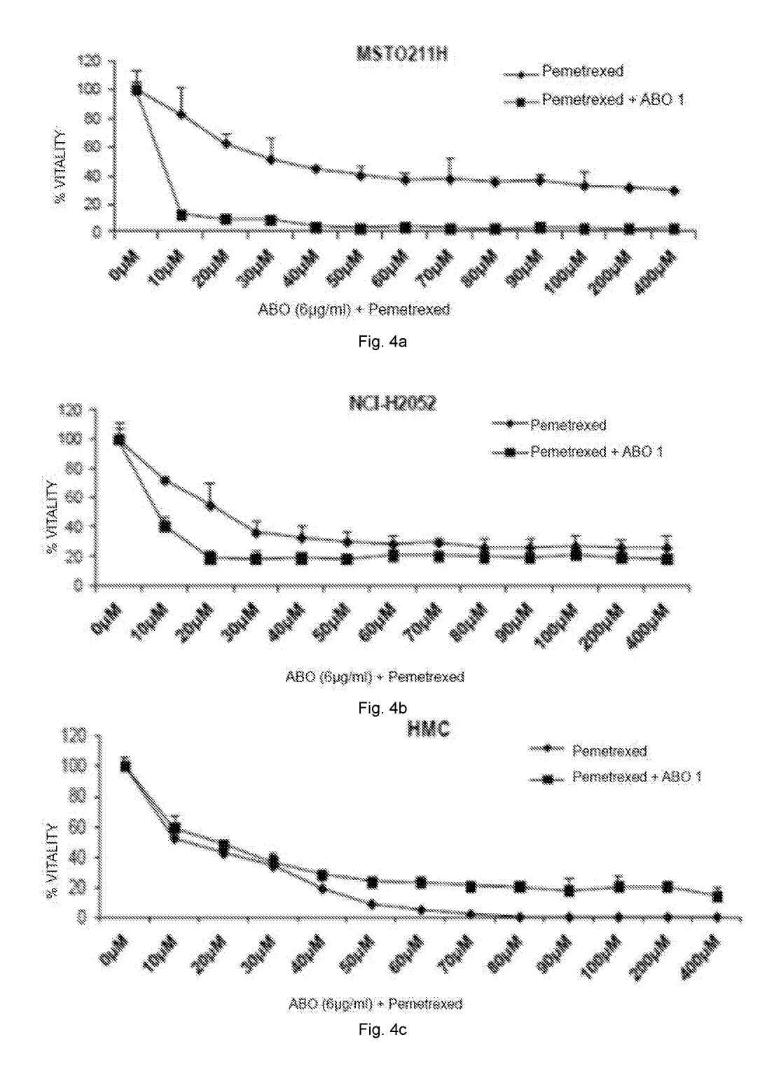

FIG. 4: Assay of cell vitality (ATPlite assay) following treatment with artichoke extract in association with Pemetrexed (PMTX) on mesothelioma cell lines MPM (FIG. 4a MSTO211H and FIG. 4b NCI-H2052) and transformed on mesothelial cells (FIG. 4c HMC). The treatment with PMTX is cytotoxic for the MPM cells and highly toxic for the non-tumour cells. The co-treatment of the cells with the extract of the invention+PMTX had a significant effect on cell vitality in MPM cell lines, whilst reducing the mortality caused by Pemetrexed in the untransformed cells (HCM). Consequently, it is clear that the extract of artichoke of the invention makes only the tumour cells sensitive to Pemetrexed.

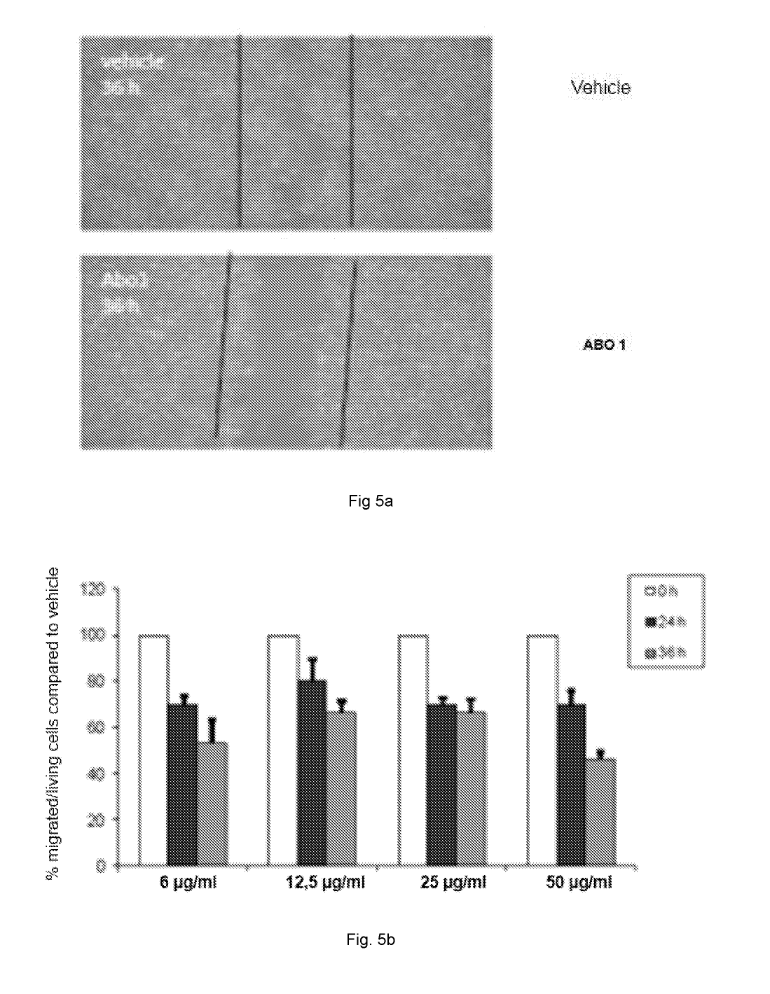

FIG. 5: Assays of wound healing on human mesothelioma cell line MSTO221H (see experimental section for the conditions).

Graph 5a shows the wound healing in control plates with just the carrier (vehicle) and with product at a concentration of 6 μg/ml, whereas image 5b shows bar charts concerning the efficacy in closing the wound (quantification of the number of cells in %) treated with the extract of the invention and with carrier at the times indicated.

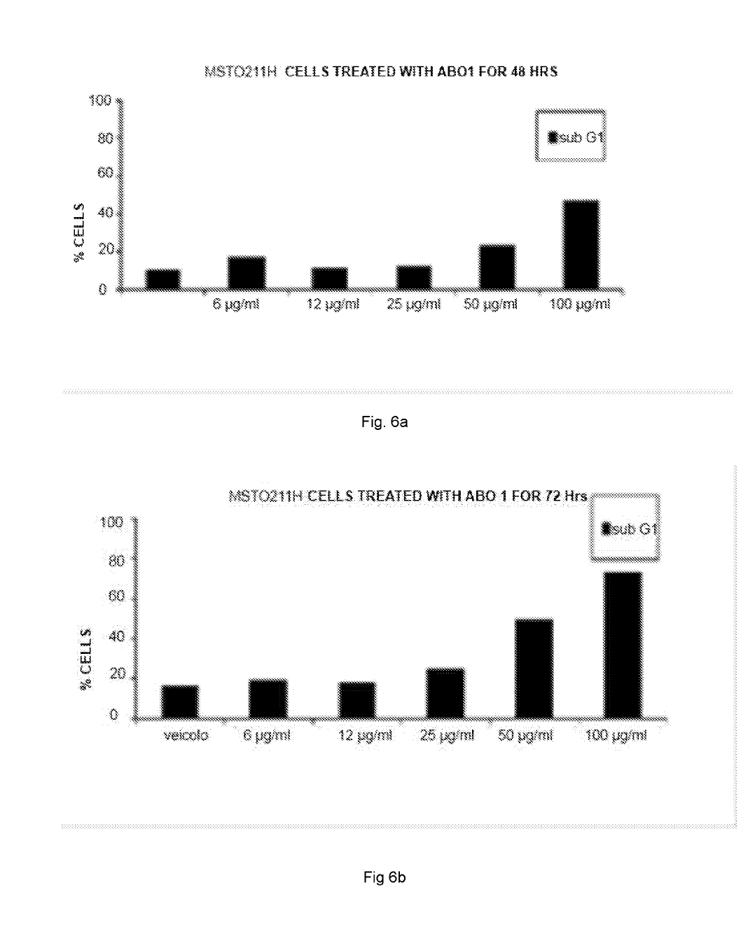

FIG. 6: Assessment of the impact of the extract of Cynara scolymus on the cell cycle (FACS method). The extract induces the death of the MPM cells (MSTO211H) by means of an increase in the % of cells in sub G1 phase, both after treatment for 48 hours (FIG. 6a) and after treatment for 72 hours (FIG. 6b).

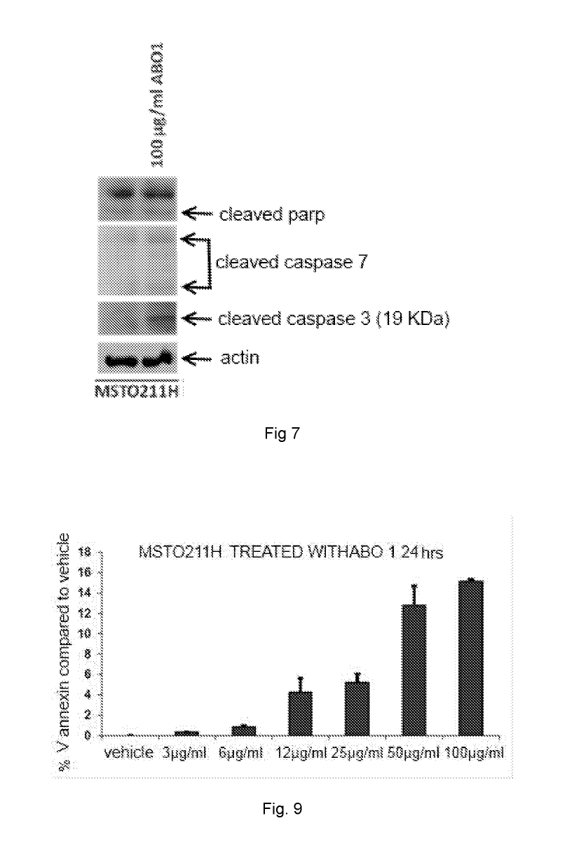

FIG. 7: Assays to assess the induction of apoptosis (Western method). The extract of the invention at the dose of 100 μg/ml induces apoptosis as demonstrated by the rise in the levels of some apoptotic markers as the cleaved form of PARP and of caspases 3 and 7 in the cell line MSTO211H.

FIG. 8: Assay to assess the induction of apoptosis by means of measurement of the level of annexin V. The extract of the invention induces apoptosis in the cell line MSTO211H, as determined by the coloration of annexin V, in a time-dependent and dose-dependent manner.

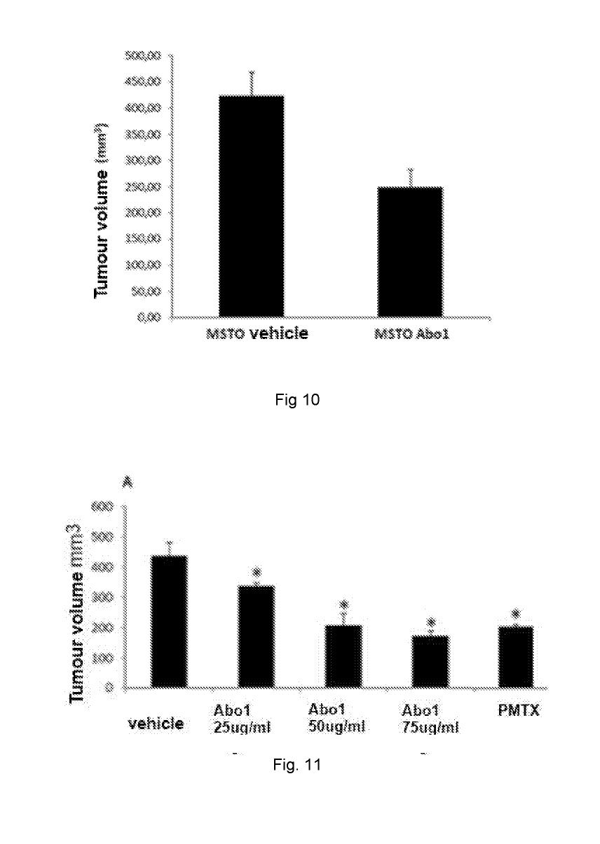

FIGS. 9 and 10 concern the assessment of the anti-tumour activity of the artichoke extract in the cell line MSTO211H, performed in nude female CD1 mice 6-7 weeks old (MPM tumour engraftment)

FIG. 9: Effect of artichoke on the engraftment of MPM cell lines

The MSTO211H were pre-treated with artichoke for 24 hours. Then, they were inoculated in nude CD1 mice. The pre-treatment with the artichoke extract influenced the engraftment of the tumour and induced a significant statistical difference (p=0.01) in the volume of the tumour.

FIG. 10: Effect of the artichoke extract on the transplantation of MPM cells. CD1 mice with xenograft of MSTO treated with growing quantities of artichoke extract for 3 weeks. A therapeutic dose-dependent effect was observed for the artichoke extract. Pemetrexed (PMTX) was used as positive control at a known therapeutic concentration. The figure shows the efficacy of the extract of the invention compared with the known therapeutic concentration of Pemetrexed*p<0.01.

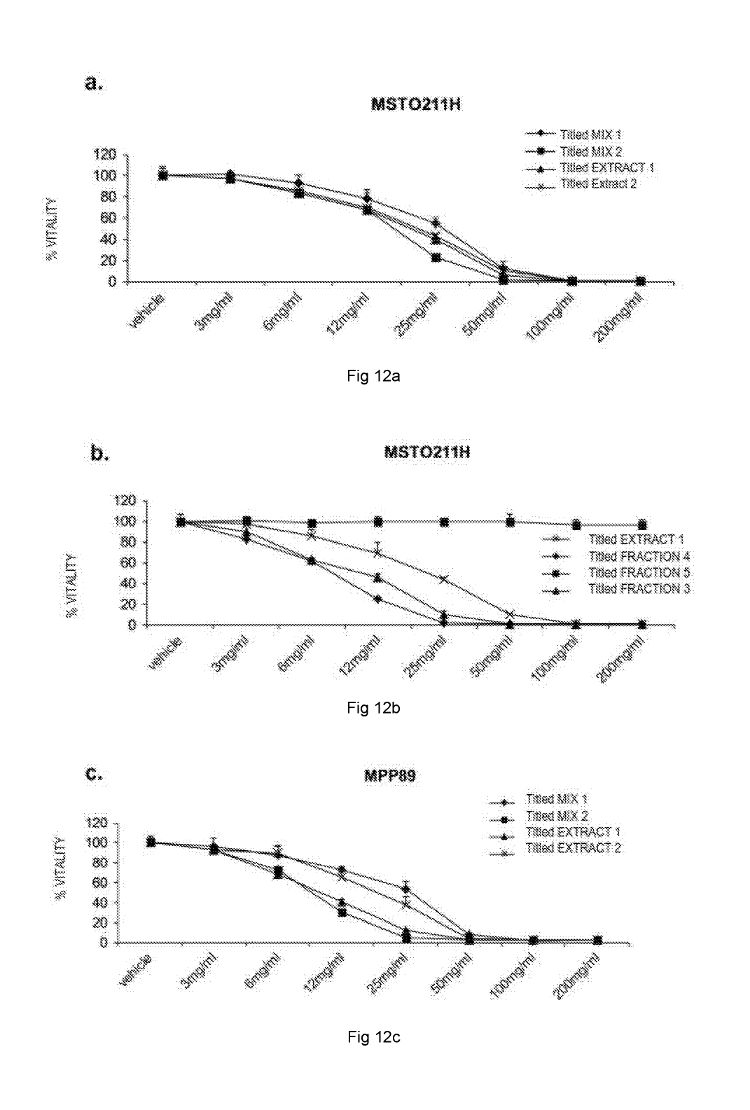

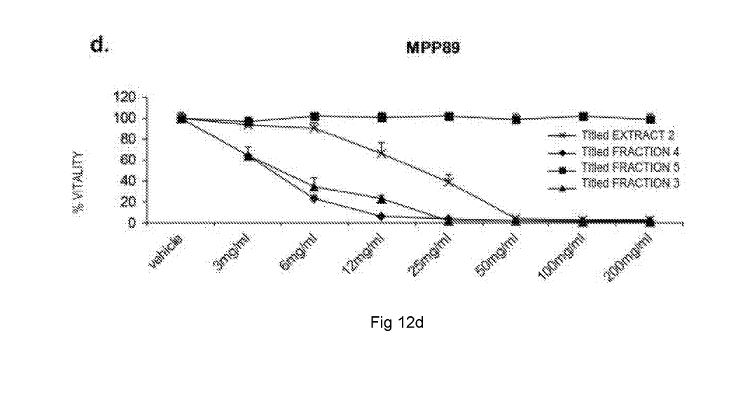

FIG. 11: Comparison of the three vitality curves with ATPlite assay on malignant pleural mesothelioma cell lines (FIGS. 11a and 11b MSTO211H, FIGS. 11c and 11d MMP-89).

In graphs a and c the assay was performed with various extracts of Cynara scolymus or with a mix of fractions of extract of Cynara scolymus, wherein total caffeoylquinic acids represent from 9% to 15% by weight of the extract or of the mixture (mix) of fractions in dry form, chlorogenic acid represents from 3.5% to 5.5% by weight of the extract or of the mixture of fractions in dry form, and cynaropicrin represents from 0.2% to 3% by weight of the extract or of the mixture of fractions in dry form.

In graphs b and d the assay was performed with a titrated extract reported also in graphs a and c, with the fractions 3, 4 and 5 as described below.

Apparently, fractions 3 and 4 are effective at least as much as an entire extract or mix titrated as described above, whereas fraction 5 alone has no effectiveness whatsoever.

### DETAILED DESCRIPTION OF THE INVENTION

The present application thus relates to an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 8% to 16% by weight of said extract or of said fraction or of said mixture (mix) in dry form, chlorogenic acid represents from 3.5% to 7% by weight of said extract or of said fraction or of said mixture in dry form, and said cynaropicrin represents from 0.2% to 4%
   or wherein
total caffeoylquinic acids represent from 25% to 48% by weight of said fraction in dry form, chlorogenic acid represents from 11% to 21% by weight of said fraction in dry form, and said cynaropicrin represents from 1% to 10% by weight of said fraction in dry form
   for use in the prevention and/or in the treatment of malignant pleural mesothelioma (MPM).

In a further embodiment, the application also relates to an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 9% to 15% by weight of said extract or of said fraction or of said mixture (mix) in dry form, chlorogenic acid represents from 3.5% to 5.5% by weight of said extract or of said fraction or of said mixture in dry form, and said cynaropicrin represents from 0.2% to 3% by weight of said extract or of said fraction or of said mixture in dry form
   or wherein
total caffeoylquinic acids represent from 25% to 35% by weight of said fraction in dry form, chlorogenic acid represents from 11% to 15% by weight of said fraction in dry form, and said cynaropicrin represents from 1% to 8% by weight of said fraction in dry form
   for use in the prevention and/or in the treatment of malignant pleural mesothelioma (MPM).

Artichoke or Cynara scolymus for the purposes of the present invention mean plants belonging to the Cynara (Cynara spp.) genus, in particular Cynara cardunculus subsp. scolymus.

For the purposes of the implementation of the present invention, the extract or its fractions may be of leaves and/or flower-heads or mixtures thereof, either fresh of dried.

The term “flower-heads” denotes the head of the flowers produced by the plant, for example the artichoke itself (part commonly used as food). The extract could be a fluid extract, or an extract lyophilised or dried by means of known drying techniques.

The extract can be obtained by means of extraction with the following solvents: water, ethanol, methanol, acetone or isopropanol, in each case in pure form or in a mixture with one another. The alcohol could be methanol, ethanol, isopropanol and is preferably ethanol. The ethanol can be used in pure form or in mixture with water at the following percentages: 96%, 90%, 85%, 80%, 75%, 70%, 65%, 60%, 55%, 50%, 45%, 40%, 35%, 30%, 25%, 20%, 15%, 10%, 5%, 1%. In a non-limiting embodiment of the invention, the solvent used for the extraction could be a mixture formed by ethyl alcohol and water in a proportion of 50:50. The fluid extract could be prepared by means of hydroalcoholic extraction by percolation/digestion of the artichoke leaves in relation to drug/solvent from 1:2 to 1:100 and preferably in a ratio of 1:10. The duration of the extraction is a duration commonly used by a person skilled in the art and could be, for example, from a minimum of 1 hour to approximately 8 hours. The temperature of extraction is normally controlled and could preferably be, for example, a temperature of approximately 50° C. The evaporation of the alcohol from the hydroalcoholic extract and the subsequent drying of the aqueous concentrate could be performed by means of lyophilisation or desiccation to provide the lyophilised extract or dry extract.

The preparation of such extracts is commonly known to a person skilled in the art and does not need to be described in particular detail in the present disclosure. For the purposes of implementing the present invention, it is possible to use any extract among those indicated above, prepared in accordance with conventional techniques.

In particular, for the purposes of the present invention, the extract could also be substituted by a fraction or by a mixture of fractions of extract of Cynara scolymus, or by a mixture of extract of Cynara scolymus and of one or more fractions of extract as described above, as long as the above-disclosed titration criteria are met. Given the variability of plant extracts, which can result from climatic conditions, environmental conditions, from differences of cultivation grounds and/or cultivation techniques, or even by the different varieties of cultivated plants, for a clinical use it is important to standardize the product and to identify standardization parameters enabling to afford a product with defined features.

The authors of the present invention have therefore characterized the extracts used in the experimenting, such as, e.g., those reported in the figures and in the experimental protocols, in order to identify parameters enabling to standardize the end product to be used in clinical practice.

The extracts used were then titrated for some active components, and were also fractionated with various techniques in order to be able to obtain fractions of extract that were titrated and titratable for the same components and be therefore able to use also individual fractions, or to mix two or more of said fractions in order to obtain an end product falling within the parameters indicated above.

The titration of the parameters is performed on dry samples, e.g., dried, dehydrated or lyophilized (freeze dried).

According to the present invention, total caffeoylquinic acids represent from 8% to 16% or from 9 to 15% by weight of said extract or of said fraction or of said mixture in dry form, such as, e.g., about 8%, 9%, 10%, 11%, 12%, 13%, 14%, 15%, 16%. The indication “about” here means that also non-integers from 8 to 16, such as, e.g., 8.1; 8.2; 8.3; etc., up to 16, are encompassed by the invention.

In a preferred embodiment, total caffeoylquinic acids represent from 11% to 13% by weight of said extract or of said fraction or of said mixture in dry form, such as, e.g., about 11%, 12%, 13% and non-integers comprised from 11 to 13.

According to the present invention, the total chlorogenic acid represents from 3.5% to 7%, or from 3.5% to 5.5% or from 4.5% to 5.5% by weight of said extract or of said fraction or of said mixture in dry form, such as, e.g., about 4.5%, 4.6%, 4.7%, 4.8%, 4.9, 5.0%, 5.1%, 5.2%, 5.3%, 5.4%, 5.5%.

According to the present invention, total cynaropicrin represents from 0.2% to 4% or from 0.2 to 3% by weight of said extract or of said fraction or of said mixture in dry form. Since cynaropicrin tends to degrade, the initial cynaropicrin content (i.e., just as the extraction or the fractioning have occurred) is preferably ranging from 2 to 3%. In any case, it is acceptable that the extract, the fraction or the mixture of fractions reach a cynaropicrin content equal to at least 0.2% at +36 months from extraction, when stored at a temperature ranging from +4° C. to +40° C., preferably at 25° C., from the extraction or from the fractioning.

The present invention also relates to a fraction of extract of Cynara scolymus wherein the percentages (percents) by weight of each one of the components indicated above are about twice those reported above, and therefore a fraction titrated in total caffeoylquinic acids, in chlorogenic acid and in cynaropicrin, wherein total caffeoylquinic acids represent from 25% to 48% or from 25% to 35% by weight of said fraction in dry form, chlorogenic acid represents from 11% to 21% or from 11% to 15% by weight of said fraction in dry form and said cynaropicrin represents from 1% to 10% or from 1% to 8% by weight of said fraction in dry form.

This fraction is suitable for all uses and implementations as compositions and kit and therapeutic method indicated in the description for the extracts, the fractions and the mixtures of fractions titrated in total caffeoylquinic acids, in chlorogenic acid and in cynaropicrin, wherein total caffeoylquinic acids represent from 8% to 16% or from 9% to 15% by weight of said extract or of said fraction or of said mixture in dry form, chlorogenic acid represents from 3.5% to 7% or from 3.5% to 5.5% by weight of said extract or of said fraction or of said mixture in dry form and said cynaropicrin represents from 0.2% to 4% or from 0.2 to 3% by weight of said extract or of said fraction or of said mixture in dry form.

According to the present invention, numerous fractions of extract of Cynara scolymus can be obtained by various methods that are indicated below. The fractions, once obtained, are titrated in total caffeoylquinic acids, in chlorogenic acid and in cynaropicrin and can then be selected, thanks to the optimal titers disclosed in the present description, fractions that can be used alone or mixtures of fractions having a concentration by weight of each one of the three titrated components as defined above.

By way of example, various fractions can be obtained by following the various methods and steps listed below:

1. Dried leaves and/or flower-heads of Cynara scolymus are fractionated, put into contact with 96° ethyl alcohol for a period ranging from 4 to 10 hours at a temperature ranging from 35 to 45° C. The alcoholic part is separated from the leaves and/or flower-heads and is subjected to filtration so as to eliminate plant residues. The clarified alcoholic solution is collected.

2. The plant residue is subjected to a further extraction with water, preferably demineralized, and the aqueous component is collected, subjecting it also to filtration so as to remove residual plant parts.

The clarified aqueous solution is collected.

3. The collected clarified (alcoholic and aqueous) solutions are mixed, obtaining an alcoholic solution ranging from 40° to 60° (in the present invention, with respect to alcohols, the sign ° denotes alcoholic grades) and is subjected to precipitation and centrifuging, with recovery of the supernatant that is subjected to filtration.

4. The precipitate obtained after supernatant removal is collected and corresponds to a first fraction of extract that can then be dried, e.g. by freeze drying, and titrated. On average, in the first fraction obtained, total caffeoylquinic acids represent about 2-3% of the total weight of the dry fraction of extract, chlorogenic acid represents about 0.2-0.6% of the total weight of the dry fraction of extract, and cynaropicrin represents about 0.1-0.3% of the total weight of the dry fraction of extract.

5. The supernatant of the 40°-50° hydroalcoholic fraction subjected to precipitation and centrifuging as described above at point 3 is concentrated under vacuum, eliminating the alcohol and therefore bringing the fraction to alcoholic 0°, the obtained concentrated aqueous solution is subjected to precipitation and centrifuging and the supernatant is subjected to filtration for clarification.

6. The precipitate obtained after supernatant removal is collected, and it corresponds to a second fraction of extract that can then be dried, e.g. by freeze drying and titrated.

On average, in the second fraction obtained, total caffeoylquinic acids represent about 1-2.5% of the total weight of the dry fraction of extract, chlorogenic acid represents about 0.05-0.1% of the total weight of the dry fraction of extract, and cynaropicrin represents about 0.3-0.5% of the total weight of the dry fraction of extract.

7. The filtrate obtained from the supernatant after the centrifuging and subjected to filtration at point 5 is an aqueous solution that is concentrated, e.g. under vacuum, and then dried, e.g. by freeze drying, and therefore corresponds to a third fraction. On average, in the third fraction obtained, total caffeoylquinic acids represent about 12-14% of the total weight of the dry fraction of extract, chlorogenic acid represents about 5-7% of the total weight of the dry fraction of extract, and cynaropicrin represents about 2.5-3.5% of the total weight of the dry fraction of extract.

8. Alternatively, the filtrate obtained from the supernatant after centrifuging and subjected to filtration at point 5 is an aqueous solution which is subjected to adsorption on high-porosity adsorbing resin.

9. The resin-adsorbed fraction is then recovered, concentrated and dried, e.g. by freeze drying, and corresponds to a fourth fraction.

On average, in the fourth fraction obtained, total caffeoylquinic acids represent about 29-32% of the total weight of the dry fraction of extract, chlorogenic acid represents about 13-15% of the total weight of the dry fraction of extract, and cynaropicrin represents about 3-4.5% of the total weight of the dry fraction of extract.

10. The fraction not adsorbed on resin is dried, e.g. by freeze drying, and corresponds to a fifth fraction.

On average, in the fourth fraction obtained, total caffeoylquinic acids represent about 0.5-0.7% of the total weight of the dry fraction of extract, chlorogenic acid represents about 0.1-0.2% of the total weight of the dry fraction of extract and cynaropicrin represents about 0.04-0.06% of the total weight of the dry fraction of extract.

According to the present invention, step 8 can be carried out on a column or on a bed with resins, able to adsorb aromatic substances or substances rich in highly unsaturated portions, or rich in alkyl or cycloalkyl groups, and to let elute non-related substances, such as many polar nonaromatic substances. The resin-adsorbed is desorbed with a suitable solvent, like, e.g., ethanol or hydroalcoholic solvents, for other uses.

Suitable chromatography resins may be, e.g., high-porosity adsorption resins of styrene-divinyl benzene copolymer, like, e.g., amberlite XAD-2, serdolit PAD-II, ADS TQ 318.

In particular, the resin will be a resin able to adsorb aromatic and/or apolar substances, like, e.g., a hydrophobic adsorbing resin, the resin consists of microspheres of a diameter of 0.2 mm-0.8 mm, with an uniformity coefficient s1.5 obtained by polymerization of Styrene and DVB without active groups, characterized by a highly porous physical structure having the parameter relative to the pore volume equal to about 1.3 mVg enabling adsorption and selective elution of organic substances, preferably of aromatic nature.

The table below reports punctual titrations data obtained on an extract of Cynara scolymus used in the experimenting reported (ABO-1) and on its fractions obtained according to the methods reported above (therefore, first, second, third, fourth and fifth fraction).

| | | | total |
|---|---|---|---|
| | Cynaropicrin | Chlorogenic acid | caffeoylquinic acids |
| | % by weight | % by weight | % by weight |
| Freeze dried | 2.05 | 5.4 | 11.85 |
| Cynara Scolymus | | | |
| extract (ABO-1) | | | |
| 1^{st} fraction | 0.24 | 0.44 | 2.48 |
| 2^{nd} fraction | 0.44 | 0.082 | 1.96 |
| 3^{rd} fraction | 3.14 | 5.89 | 13.37 |
| 4^{th} fraction | 6 | 32.01 | 19.68 |
| 5^{th} fraction | 0.05 | 0.61 | 0.82 |

By way of example, therefore, the mixture of fractions may be a mixture comprised of fraction 1 by about 12%, of fraction 2 by about 6%, and of fraction 3 by about 82%, or a mixture comprised of fraction 1 by about 12%, of fraction 2 by about 6%, of fraction 4 by about 55% and of fraction 5 by about 27%.

By titrating the obtained fractions, a person skilled in the art will certainly know how to reconstitute a mixture of fractions or to supplement an extract particularly poor in pharmaceutically active ingredients to obtain a compound with the optimal titration indicated above.

Fractions 3 and 4 are useful as such, as is apparent from data reported in FIG. 11. In the present invention the [Italian] term “active pharmaceutical ingredient” is equivalent to the English term “Active pharmaceutical ingredient” (API).

The term “active pharmaceutical ingredient” can also be replaced by the term “active ingredient” (or “active principle”) meant as set of molecules with pharmacological activity.

a. an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein total caffeoylquinic acids represent from 8% and 16% or from 9% to 15% by weight of said extract or of said fraction or of said mixture in dry form, chlorogenic acid represents from 3.5% to 7% or from 3.5% to 5.5% by weight of said extract or of said fraction or of said mixture in dry form, and said cynaropicrin represents from 0.2% to 4% or from 0.2 to 3% by weight of said extract or of said fraction or of said mixture in dry form (above-described detailed embodiments included);

b. a fraction of extract of Cynara scolymus titrated in total caffeoylquinic acids, in chlorogenic acid and in cynaropicrin, wherein total caffeoylquinic acids represent from 25% to 48% or from 25% to 35% by weight of said fraction in dry form, chlorogenic acid represents from 11% to 21% or from 11% to 15% by weight of said fraction in dry form, and said cynaropicrin represents from 1% to 10% or from % to 8% by weight of said fraction in dry form.

In accordance with the present invention, the extract of Cynara scolymus or a fraction thereof or a mixture of fractions thereof titrated in total caffeoylquinic acids, in chlorogenic acid, and in cynaropicrin, as described in detail above and in the claims, could be used as active pharmaceutical ingredient for the prevention and/or the treatment of malignant pleural mesothelioma MPM.

Such diseases can be, for example and as noted in the literature, diseases of the inflammatory and/or pre-tumour and/or tumour type.

In particular, the present invention could be suitable for the treatment of populations exposed to asbestos and therefore at risk of malignant pleural mesothelioma and for the reduction of the production of mesotheline in such populations of patients.

The experimental data presented below and in the figures, obtained on mesothelioma tumour cell lines, also show that the extract of Cynara scolymus or a fraction thereof or a mixture of fractions thereof titrated as disclosed in the present description can be advantageously associated with one or more anti-tumour drugs, thus increasing, also synergically, the anti-tumour efficacy of the drugs themselves. Thus, in accordance with an embodiment of the present invention, the extract of Cynara scolymus, a fraction thereof or a mixture of fractions thereof as described and claimed here can be used in the prevention and/or in the treatment of malignant pleural mesothelioma (MPM) in association with one or more compounds with anti-tumour activity (anti-tumour compounds).

In accordance with an embodiment, the compound with anti-tumour activity can be a chemotherapeutic agent and can be selected from the group comprising cisplatinum, doxorubicin, pemetrexed, methotrexate, vinorelbine, gemcitabine and taxol.

The present invention also comprises the use of extract of an extract of Cynara scolymus or a fraction of extract of Cynara scolymus or of a mixture of said extract with one or more of said fractions or of a mixture of said fractions titrated according to what disclosed in the present description in association with one or more chemotherapeutic agents for the prevention and/or the treatment of malignant pleural mesothelioma.

In particular, the extract, the fraction or the mixture according to the present invention will be titrated in total caffeoylquinic acids, in chlorogenic acid and in cynaropicrin, wherein total caffeoylquinic acids represent from 8% to 16% or from 9% to 15% by weight of said extract or of said fraction or of said mixture (mix) in dry form, chlorogenic acid represents from 3.5% to 7% or from 3.5% to 5.5% by weight of said extract or of said fraction or of said mixture in dry form, and said cynaropicrin represents from 0.2% to 4% or from 0.2% to 3% by weight of said extract or of said fraction or of said mixture in dry form, or the fraction will be a fraction wherein total caffeoylquinic acids represent from 25% to 48% or from 25% to 35% by weight of said fraction in dry form, chlorogenic acid represents from 11% to 21% or from 11% to 15% by weight of said fraction in dry form, and said cynaropicrin represents from 1% to 10% or from 1% to 8% by weight of said fraction in dry form.

The detailed description of the titration ranges of total caffeoylquinic acids, chlorogenic acid and cynaropicrin provided above also applies to this specific embodiment.

The association with one or more chemotherapeutic agents may be a concomitant or sequential association, or the active pharmaceutical ingredient of the invention and the chemotherapeutic agents can be administered at the same time (in a single administration or in separate administrations) or over a period of time of a few minutes, or can be administered sequentially or at different times, separated from one another by more than a few minutes, over the course of the day or the period of therapeutic treatment.

The administration regime will be determined by the treating doctor in accordance with the sex, the age, the state of disease, the weight and the history of the patient. Both alone and in association, as described above, the treatment can be preventative, for example in cases of infection known to have possible tumourigenic effects such as those indicated above, or in the case of ablation of tumours so as to prevent said tumours from reforming.

The active pharmaceutical ingredient of the present invention can be formulated in compositions that can be used for the same objectives as described above.

The present invention therefore further relates to a composition comprising, as active pharmaceutical ingredient, an extract of Cynara scolymus, a fraction thereof or a mixture of fractions thereof or a mixture of said extract with one or more of said fractions thereof, titrated in total caffeoylquinic acids, in chlorogenic acid and in cynaropicrin as described above and as claimed, and a carrier and/or diluent and/or excipient for use in the prevention and/or in the treatment of malignant pleural mesothelioma (MPM).

The composition may comprise the active pharmaceutical ingredient of the invention as defined here, in a lyophilised, dry or fluid form.

As already indicated, the extract and the fractions thereof can be obtained by extraction of the leaves of artichoke or of the flower-heads of artichoke or of mixtures of the aforementioned parts, whether fresh or dried, according to the methods described above.

According to the present invention, the composition as defined above can be used for the prevention and/or the treatment of malignant pleural mesothelioma.

The composition could, e.g., contain as sole active ingredient Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein total caffeoylquinic acids represent from 8% to 16% or from 9% to 15% by weight of said extract or of said fraction or of said mixture in dry form, chlorogenic acid represents from 3.5% to 7% or from 3.5% to 5.5% by weight of said extract or of said fraction or of said mixture in dry form and said cynaropicrin represents from 0.2% to 4% or from 0.2% to 3% by weight of said extract or of said fraction or of said mixture in dry form.

Alternatively, the composition could comprise as sole active ingredient a fraction of extract of Cynara scolymus wherein total caffeoylquinic acids represent from 25% to 48% or from 25% to 35% by weight of said fraction in dry form, chlorogenic acid represents from 11% to 21% or from 11% to 25% by weight of said fraction in dry form, and said cynaropicrin represents from 1% to 10% or from 1% to 8% by weight of said fraction in dry form.

Furthermore, the composition could comprise excipients suitable for the type of formulation selected.

A person skilled in the art will be able to readily identify the best formulations.

Merely by way of example, the composition could be made in form of hard gelatine capsule and contain from 30% to 60% of active ingredient as defined above in lyophilised form and from 70% to 40% of suitable pharmacologically inert excipients (like, e.g., microcrystalline cellulose).

The capsule could be, e.g., a capsule having a final weight of 300-500 mg.

Since the active compound as described herein could be in a lyophilised, dried or fluid form, a person skilled in the art could readily make pharmaceutical compositions suitable for the selected use.

According to a non-limiting example, the present invention, the composition as defined here can be used for the prevention and/or the treatment of malignant pleural mesothelioma.

Therefore, the invention further relates to a composition comprising, beside the active pharmaceutical ingredient of the invention, one or more compounds with anti-tumour activity (anti-tumour compounds) for use in the prevention and/or the treatment of malignant pleural mesothelioma.

In accordance with an embodiment, such compounds with anti-tumour activity (anti-tumour compounds) may be chemotherapeutic agents selected, for example, from the group comprising cisplatinum, doxorubicin, pemetrexed, methotrexate, vinorelbine, gemcitabine and taxol.

The composition of the invention can be formulated in unit doses or in a dosable manner by the treating doctor for the purpose of also enabling therapies adapted to the individual needs of each patient.

The present invention thus envisages the use of compositions comprising as sole active pharmaceutical ingredients the active pharmaceutical ingredient of the invention, optionally in association with one or more further active pharmaceutical ingredients having anti-tumour activity for the prevention and/or the treatment of malignant pleural mesothelioma.

Such further active pharmaceutical ingredients may be, for example, chemotherapeutic compounds.

The association with the one or more chemotherapeutic agents may be a concomitant or sequential association, or the active pharmaceutical ingredient and the chemotherapeutic agents can be administered at the same time (in a single administration or in separate administrations) or over a period of time of a few minutes, or can be administered sequentially or at different times, separated from one another by more than a few minutes, over the course of the day or the period of therapeutic treatment.

The administration regime will be determined by the treating doctor in accordance with the sex, the age, the state of disease, the weight and the history of the patient. The treatment described can be preventative, in the case of ablation of tumours, so as to prevent said tumours from reforming.

In the composition as described above (consisting in the active pharmaceutical ingredient of the invention and at least one pharmaceutically acceptable excipient or adjuvant, optionally in association with one or more anti-tumour agents) at least one pharmaceutically acceptable excipient or adjuvant may be selected among excipients or adjuvants technically used in common pharmaceutical or cosmetic practice or in the food industry. The excipients used may belong to the category of diluents, solubilisers, disintegrators, binders, lubricants, surfactants, slip agents and anti-adherents.

If necessary, the composition may also contain flavourings, colorants and preservatives used commonly in the pharmaceutical, cosmetic and food industries. The compositions can be in any formulation considered suitable by a person skilled in the art preparing formulations intended for oral administration (for example powders, granulates, capsules in hard or soft gelatine, tablets, syrups, drops, solutions and oral emulsions), inhalation (for example aerosols, liquid and powder sprays), topical administration (gels, ointments, emulsions, pastes, foams, anhydrous solid forms for topical application, and patches) and parenterally in accordance with the techniques currently used and known to a person skilled in the art (for example for subcutaneous use, intramuscular use, intravenous use or intradermal use). In all formulations, the use of technological excipients or adjuvants is determined by selecting the substances to be used on the basis of those used commonly in pharmaceutical practice.

In the preparation of formulations based on the active pharmaceutical ingredient of the invention in association or not with agents having anti-tumour activity, a person skilled in the art could use any of the excipients deemed useful in accordance with the prior art in order to obtain a stable preparation suitable for use in therapy. By way of example, in the category of diluents, it is possible to use diluents in solid formulations, such as sugars, polyalcohols (for example lactose, mannitol, sorbitol), cellulose, salts of inorganic acids (for example dibasic calcium phosphate), salts of organic acids (such as citrates, carbonate and bicarbonate titrates in the form of salts of sodium, potassium and calcium), or diluents in liquid forms, such as water, edible oils for oral use (sunflower oil, olive oil, corn oil, sweet almond oil, nut oil) or used in topical formulations (jojoba oil, short-chain, medium-chain or long-chain triglycerides), polyalcohols (glycerin, propylene glycols, hexylene glycol).

In the category of the disintegrators, it is possible to use, for example, natural or modified starches (corn starch, rice starch, potato starch), croscaramellose sodium, glycolate sodium starch, crospovidone; possible binders that can be used include natural products of the rubber type (guar gum, xanthan gum, gum arabic), sucrose and synthesis products, including polyvinyl pyrrolidone and semi-synthetic derivatives of cellulose.

The use of stearic acid and salts thereof, including the salt of magnesium, polymers of ethylene glycol, triglycerides and natural or synthetic waxes as lubricants has proven to be effective.

The surfactants are used to make one or more active ingredients contained in the formulations forming the basis of the invention more soluble or washable with water, these active ingredient acting alone or carried by one or more diluents. For example, sorbitan esters, sorbitan polyoxyethylene esters, sucrose esters and sodium lauryl sulphate can be cited.

The slip agents may be selected for example from colloidal silica, precipitated silica, whereas the anti-adherents that can be used include, for example, talc or starch.

In the preparation of injectable formulations, it is possible to choose those excipients that allow effective solubilisation or dispersion of the active substance(s). By way of example, together with water, other hydrosoluble carriers such as polyalcohols and salts of organic or inorganic acids can be used for the purpose of obtaining pH and osmolarity suitable for the administration by means of injections. In particular cases, it will be possible to use non-hydrosoluble carriers, such as oils, or substances of synthesis commonly approved for injective use.

A person skilled in the art can prepare all the formulations using the common preparation schemas known to him/her.

Merely by way of example, a formulation in capsules can be prepared conveniently by grinding beforehand the active pharmaceutical ingredient of the invention, mixing in a common mixer the powder obtained together with one or more other anti-tumour agents and the excipients selected to prepare the formulation, for example a diluent, a disintegrator, a lubricant and a slip agent selected from those mentioned above or available on the market and approved for oral use.

In the case of a tablet, it could be necessary to granulate some or all of the mixture with a binding agent dissolved beforehand in water or introduced in mixture and using the water as an adjuvant of the process of granulation in accordance with the prior art.

The granulate may be dried, sieved and further mixed with other powders for the purpose of obtaining a mixture suitable for obtaining tablets in accordance with that known to a person skilled in the art.

In the case of parenteral use, the composition may also be provided with the active pharmaceutical ingredient of the invention and one or more anti-tumour agents in separate containers conveniently miscible in accordance with specific operational requirements.

For the purpose of facilitating the use of the compositions described here, these can be presented in the form of unit doses containing the active pharmaceutical ingredient of the invention and optionally one or more anti-tumour agents effective for a preventative and/or therapeutic use of malignant pleural mesothelioma.

The present invention further relates to a kit for the concomitant or sequential administration of the active pharmaceutical ingredient of the invention and one or more compounds with anti-tumour activity for use in the prevention and/or in the treatment of malignant pleural mesothelioma, said kit comprising one or more aliquots of the active pharmaceutical ingredient of the invention as defined in the present description, and one or more aliquots of one or more compounds having anti-tumour activity.

Alternatively, the kit may comprise one or more aliquots of the composition containing, as sole active pharmaceutical ingredient, the active pharmaceutical ingredient of the invention as defined in the present description and one or more aliquots of one or more compounds having anti-tumour activity.

As described above, such compounds can be chemotherapeutic agents selected for example from the group comprising cisplatinum, doxorubicin, pemetrexed, methotrexate, vinorelbine, gemcitabine and taxol.

According to the present invention, the pathology on which the compositions described herein exert their therapeutical activity is represented by malignant pleural mesothelioma.

Lastly, the present description also concerns a therapeutic method for the prevention and/or the treatment of malignant pleural mesothelioma comprising the step of administering to an individual in need of it a therapeutically active quantity of the active pharmaceutical ingredient or of a pharmaceutical composition comprising, as sole active pharmaceutical ingredients, the active pharmaceutical ingredient of the invention, optionally in association with one or more anti-tumour compounds.

The method forming the basis of the present invention can be carried out by administering to a subject who presents a pre-tumour and/or tumour pathological condition of malignant pleural mesothelioma, therapeutically effective doses of the active pharmaceutical ingredient as defined here, optionally in association with one or more anti-tumour drugs; or by administering therapeutically effective doses of the composition as defined here, optionally further comprising one or more anti-tumour drugs, or by administering the extract and one or more anti-tumour drugs using the kit as defined here.

The administration, as described above, can be performed concomitantly or sequentially in accordance with the administration regime selected by the doctor.

In a preferred embodiment of the invention, the pathology is represented by malignant pleural mesothelioma.

Numerous experimental data have been reported that demonstrate the efficacy of the extract according to the present invention.

### Used Cell Lines

MSTO211H Human lung biphase mesothelioma cell line with constitutively activated STAT3. Available from ATCC #CLR-2081

Human mesothelioma cell line, established from the pleural spill of a human of 62 years of age with mesothelioma (biphase malignant) who had not had any prior therapy. Cell line MSTO211H expresses high levels of pStat3). (Tsao et al. Inhibition of c-Src expression and activation in malignant pleural mesothelioma tissues leads to apoptosis, cell cycle arrest, and decreased migration and invasion. MolCancerTher 2007; 6:1962-1972.)
NCI-H2052 Human mesothelioma cell line. This cell line expresses pSTAT3 (Tsao et al. Inhibition of c-Src expression and activation in malignant pleural mesothelioma tissues leads to apoptosis, cell cycle arrest, and decreased migration and invasion. MolCancerTher 2007; 6:1962-1972.) Available from ATCC #CLR-5915
NCI-h28: Human stage-4 mesothelioma cell line. Available from ATCC#CRL-5820
MPP-89 Human mesothelioma cell line. Available from CABRI, access number ICLC HTL00012

The following examples show how the extract of Cynara scolymus of the present invention is able to:

reduce the vitality in mesothelioma cells (MSTO211H, MPP-89, NCI-H2052, NCI-H28) in a dose-dependent manner, acting less strongly on non-transformed mesothelial cells (HMC);

reduce the ability to form colonies in assays of clonogenic survival over the same cell lines,

induce cell death of malignant mesothelioma cells MM in apoptotic assays;

inhibit the migration and the proliferation of MM cells in wound healing assays;

sensitise the MM cells with successive treatments with a chemotherapeutic agent, such as pemetrexed;

induce damage in the DNA of MM cells whilst not inducing damage to the DNA of HMC cells;

reduce the ability of tumour transplantation with MSTO cells on cells pre-treated with the extract;

have a dose-dependent effect in the treatment of xenotransplantation of MSTO.

### Examples

### 1. Methods

### 1.1. Cell Lysis and Western Blotting.

The cells were lysed in ice for 30 min in lysis buffer NP40 (50 mM Tris-HCl pH 7.4, 150 mM NaCl, 1% NP-40, 1 mM EGTA, 1 mM EDTA) complemented with inhibitors of protease and phosphatase (5 mM PMSF, 3 mM NaF, 1 mM DTT, 1 mM NaVO4). Equal amounts of total extracts of protein (30 μg) were broken down by means of denaturing electrophoresis (SDS-PAGE) in 8% polyacrylamide gel and transferred for 2 hours on nitrocellulose membrane. The membranes were blocked with a 5% solution of milk dissolved in TBS-Tween_20 0.05% for 1 hour and incubated with the specific primary antibodies. The following primary antibodies were used: anti-beta actin (A-2228, SIGMA), anti-pSTAT3 (Tyr-705) (sc8059, Santa Cruz) and anti-STAT3 (sc7179, Santa Cruz). The secondary antibodies were peroxidase-conjugated (Santa Cruz), and ECL reagents (Amersham, GE Healthcare, Piscataway, N.J., USA) were used for the chemiluminescence.

1.2. Treatment of the Cell Lines of MPMs and of Normal Commercial Mesothelial Cells (HMC) with Extract of Cynara Scolymus.

The cell lines of MPMs (MSTO-211H, NCI-H28, NCI-H2052, MPP89) were acquired from ATCC (Rockville, Md.) whilst the HMCs (Human Mesothelial Cells) were acquired from Tebu-Bio (France). All the lines were grown in monolayers at 37° C. and at 5% of CO2 in specific culture media. The artichoke extract was dissolved conveniently in a solution of water for injectable solutions and ethanol in a ratio of 1:1 at an initial concentration of 30 mg/ml. To test the anti-tumour property, the product was then added directly in the medium of the various cell lines using various concentrations and various times, as shown in the drawings.

### 2. Assay of Clonogenicity on Cells of Malignant Pleural Mesothelioma (MPM)

MPM cells (MSTO211H, NCI-H28; MPP-89; NCI-H2052) were seeded at 200 cells per well and were treated with various growing concentrations (control just with carrier; 12.5 μg/ml; 25 μg/ml; 50 μg/ml; 100 μg/ml, 200 μg/ml) of extract of Cynara scolymus in accordance with the present description. Each point was plated in duplicate in the 6-well multiwell. The colonies formed were stained with violet crystal 15-21 days later. The colony formation assay, also known as clonogenic assay, is a technique used to assess the efficacy of anti-tumour compounds in terms of the ability of the tumour cells to form colonies from a single cell. A colony is considered to be a group of 50 or more cells (clones) originating from a single cell.

The results of the experiment, shown in FIGS. 1a-1d, show the dose-dependent ability of the extract of the invention to inhibit, in a dose-dependent manner, the formation of colonies in all the MPM cell lines assayed.

### 3. ATPlite™ Cell Vitality Assay

The vitality of various cell lines following exposure to the extract of the invention at various concentrations was assessed using the ATPlite™ assay (Perkin Elmer) in accordance with the producer's instructions. Where indicated, the term “carrier” refers to a solution of water for injectable solutions and ethanol at a concentration of 1:1 used in the same volumes used for the treatments.

ATPLite™ is a system for monitoring the levels of adenosine triphosphate (ATP) based on the activity of firefly (Photinus pyralis) luciferase. This luminescence assay is an alternative to colorimetric, fluorometric and radioisotopic tests for the quantitative evaluation of the proliferation of cultured mammalian cells subjected to treatment with possible substances contained in the culture medium. The monitoring of ATP is used in fact to evaluate the cytostatic and anti-proliferative effects of a vast range of drugs, modifiers of the biological response, and biological compounds. The ATPLite™ test system is based on the production of light caused by the reaction with addition of ATP luciferases and D-luciferin. The light emitted is proportional to the concentration of ATP within certain limits. The quantity of ATP in cells correlates with the cell vitality.

The cell vitality of various types of MPM cell lines (MSTO211H, MPP89, NCI-H28) and of HMC cells (untransformed mesothelial cells provided by willing donors) were assayed following treatment with various concentrations of extract according to the invention (control just with carrier; 12.5 μg/ml; 25 μg/ml; 50 μg/ml; 100 μg/ml, 200 μg/ml).

The graph in FIG. 2, which shows the results of the assay, shows that the extract is able to significantly reduce cell vitality in a dose-dependent manner.

The effects on cell vitality were also tested on untransformed mesothelial cells (HMCs), towards which the extract forming the basis of the invention demonstrated lower cytotoxicity compared with the tumour lines (FIGS. 6A-6C) (FIG. 6A-6C).

### 4. Assays of Cell Vitality in Co-Treatment with Chemotherapeutic Agents

Cell lines MSTO211H and NCI-H2052 were used to evaluate the effects of the association of extract of Cynara scolymus+anti-tumour drug.

The assay shown in FIG. 4 (a-c) was performed using ATPlite™ assay (Perkin Elmer) in accordance with the producer's instructions.

A solution of water for injectable solutions and ethanol at a concentration of 1:1 was used in the same volumes used for the treatments.

### Reagents:

pemetrexed (Alimta, Lilly) diluted in accordance with the producer's instructions.
4.1 Association of Extract of Cynara Spp. and Pemetrexed with ATPlite™ Assay

FIG. 4 show the vitality curve for MSTO211H after 72 h of treatment with pemetrexed and pemetrexed in association with extract of Cynara spp. Graph A shows the treatment with the extract at non-cytotoxic dose (6 μg/ml) and pemetrexed for the MSTO211H cells, whereas graph B shows the treatment with the extract at non-cytotoxic dose (6 μg/ml) and with pemetrexed (various concentrations) for NCI-H2052 cells, and graph C shows the treatment with the extract at non-cytotoxic dose (6 μg/ml) and with pemetrexed (various concentrations) for untransformed HMC cells. The concentrations of the assayed compound are plotted on the abscissa, whereas the cell vitality expressed in percentage is plotted on the ordinate.

FIGS. 4 a and b show how the treatment with extract sensitises the tumour lines to the treatment with pemetrexed. In the curve with double treatment, it is clear how just a concentration of pemetrexed of 10 μM is sufficient to lower the cell vitality of the tested lines. It is interesting to note that, in the non-tumour line, the extract has a protective effect towards pemetrexed.

### 5. Wound Healing Assay

The wound healing assay (FIG. 16) is simple, inexpensive, and one of the first methods developed for studying directional cell migration in vitro. This method mimics cell migration during wound healing in vivo. The basic steps involve creating a “wound” in a cell monolayer, then monitoring a specific zone of the “wound” by capturing images at the beginning and at regular intervals during the cell migration necessary to close the “wound”. The MSTO211H cells cultivated with a confluency of 95% were seeded in 6-well plates and the “wound” (or cut) was made with a puncture by 10-microlitre sterile pipette to remove the cells. Digital micrographs were produced after the wounds at the indicated times. The final bar chart shows the efficacy of closure of the cut (quantification number of the cells in %) treated with carrier or ABO 1 at the indicated times.

### 6. Assay to Assess the Induction of Apoptosis

See Figures (6-8)

### 6.1 Western Blotting

The same technique as described in point 1.1 was used, and the following primary antibodies were used: anti-beta actin (A-2228, SIGMA), anti-caspase-3 (31A1067, Alexis), anti-caspase-7 (#9492, Cell Signalling) and anti-PARP (#9542S, Cell Signalling).

### 6.2 FACS Analysis and PI Staining and PI/Annexin V Staining Analyses

For the purpose of determining the effect of the extract of the invention on the cell cycle, a FACS analysis was performed.

For staining with propidium iodide (PI), the cells were seeded in 6-well plates at a density of 10⁴ cells/ml. After 24 h, the tumour cells were treated with indicated concentrations of the extract of the invention for various time intervals. The cells were collected in suspension and the adhered cells were washed in PBS, fixed with frozen ethanol (70% v/v) and stored at −20° C. For the analyses, the cells were washed in PBS 1× and suspended in a solution of PBS 1Z, PI (25 mg/ml) and Rnase A (200 mg/ml).

For the PI/annexin V double staining, the treated cells were collected and resuspended in binding buffer (HEPES pH 7.4, CaCl2 2.5 mM, NaCl 140 mM). Aliquots of cells were incubated for 15 min with annexin V FITC and PI (5 mg/mL) (Invitrogen).

During all the FACS analyses, 10⁵ events were analysed for each sample. The flow cytometry analyses were performed on a GuavaEasyCyte 8HT (Millipore) flow cytometer.

As can be seen in FIG. 8, the extract of the invention induces apoptosis in MSTO211H cells, as determined by the annexin V staining, in a time-dependent and dose-dependent manner.

### 7. Transplantation of Tumour Cells Treated or Untreated with the Extract of the Invention

### Description of the First Engraftment Experiment.

The MSTO211H cells were treated with artichoke at the concentration of 50 μg/ml for 24 hours. A suspension of 2×10⁶ of cells in PBS/Matrigel (BD Biosciences) was collected and inoculated in the right hip of nude female mice 4 weeks old. The volume of the tumours was monitored twice a week up to the 21^{st} day. The mice were sacrificed and the masses removed.

### 8. Transplantation of Tumour Cells in Mice and Treatment with Cynara scolymus and Pemetrexed

### Description of the Second Engraftment Experiment.

The cells were expanded prior to the implantation and were evaluated in terms of their vitality and contamination, that is to say were counted and resuspended in PBS at a concentration of 20×10⁶/ml. Matrigel was added to the suspension to obtain a final concentration of 10×10⁶ cells/ml of PBS Matrigel 1/1. The MSTO cells were inoculated under the skin in 48 mice.

When the tumour reached an average volume of 60 mm³, the mice were divided into 8 groups formed by 6 animals per group receiving different treatments.

Two groups received artichoke in drinking water for 7 days of the week during a period of three weeks; the other groups received pemetrexed intraperitoneally for 5 days of the week during a period of 3 weeks.

The groups have been outlined in this way in Table 5 below:

| | no. | | | | | | | | | | | | |
|---|---|---|---|---|---|---|---|---|---|---|---|---|---|
| | ani- | cell | no. | path- | | treatm. | start of | administrat. | treatm. | treatm. | start of | administrat. | treatm. |
| | mals | line | cells | way | volume | A | treatm. | method | regime | B | treatm. | method | regime |
| Group | 6 | MSTO | 2 × 10⁶ | SC | 0.2 | | | | | Cynara | after | OS | drinking |
| 1 | | | | | (matrigel) | | | | | extract | tumour | | water |
| | | | | | | | | | | 20 μg/ml | appear- | | |
| | | | | | | | | | | | ance | | |
| Group | 6 | MSTO | 2 × 10⁶ | SC | 0.2 | | | | | Cynara | after | OS | drinking |
| 2 | | | | | (matrigel) | | | | | extract | tumour | | water |
| | | | | | | | | | | 50 μg/ml | appear- | | |
| | | | | | | | | | | | ance | | |
| Group | 6 | MSTO | 2 × 10⁶ | SC | 0.2 | | | | | Cynara | after | OS | drinking |
| 3 | | | | | (matrigel) | | | | | extract | tumour | | water |
| | | | | | | | | | | 75 μg/ml | appear- | | |
| | | | | | | | | | | | ance | | |
| Group | 6 | MSTO | 2 × 10⁶ | SC | 0.2 | | | | | | | | |
| 4 | | | | | (matrigel) | | | | | | | | |
| Group | 6 | MSTO | 2 × 10⁶ | SC | 0.2 | Peme- | after | IP | 5 days in | Cynara | after | OS | drinking |
| 5 | | | | | (matrigel) | trexed | tumour | | succes- | extract | tumour | | water |
| | | | | | | (100 | appear- | | sion | 20 μg/ml | appear- | | |
| | | | | | | mg/kg) | ance | | | | ance | | |
| Group | 6 | MSTO | 2 × 10⁶ | SC | 0.2 | Peme- | after | IP | 5 days in | Cynara | after | OS | drinking |
| 6 | | | | | (matrigel) | trexed | tumour | | succes- | extract | tumour | | water |
| | | | | | | (100 | appear- | | sion | 50 μg/ml | appear- | | |
| | | | | | | mg/kg) | ance | | | | ance | | |
| Group | 6 | MSTO | 2 × 10⁶ | SC | 0.2 | Peme- | after | IP | 5 days in | Cynara | after | OS | drinking |
| 7 | | | | | (matrigel) | trexed | tumour | | succes- | extract | tumour | | water |
| | | | | | | (100 | appear- | | sion | 75 μg/ml | appear- | | |
| | | | | | | mg/kg) | ance | | | | ance | | |
| Group | 6 | MSTO | 2 × 10⁶ | SC | 0.2 | Peme- | after | IP | 5 days in | | | | |
| 8 | | | | | (matrigel) | trexed | tumour | | succes- | | | | |
| | | | | | | (100 | appear- | | sion | | | | |
| | | | | | | mg/kg) | ance | | | | | | |

SC = subcutaneous
treatm. = treatment
administrat. = administration
IP = intraperitoneal
OS = oral

With appearance of progression of the tumour (that is to say when the tumour reached 60 mm³), treatment was started with Abo1 and pemetrexed administered as follows: pemetrexed at a dose of 100 mg/Kg in 88 ml/mouse for 5 consecutive days intraperitoneally), artichoke extract in drinking water at concentrations of 25, 50 and 75 micrograms/ml and measured on alternate days for a period of 3 weeks. The mice were monitored daily to evaluate any signs; body weight was monitored twice weekly.

At the end of the experiment (42 days after inoculation), the tumour masses were collected and fixed in 10% formalin (transferred after 24 hours to 70% ethanol).

The tumour diameters were measured twice weekly using a Mitutoyo caliper.

### 9. Determination of Chlorogenic Acid and Cynaropicrin in Cynara scolymus

Sample preparation: weigh 0.25 g of lyophilised extract (0.5 g of ground leaves) and extract with 50 ml of 75% MeOH/0.1% HCOOH under ultrasound for 15 min, protected from light. Centrifuge and decant in a 100 ml volumetric flask. Repeat on the residue a second extraction under the same conditions. Centrifuge and decant in the same 100 ml flask. Bring to volume the reunited organic extracts at 20° C. with the same extraction solvent. Filter over a 0.45 μm cellulose acetate filter and inject into a UHPLC or HPLC system.

### Chromatographic Conditions (UHPLC):

Column: Poroshell 120 EC-C18, 3×100 mm 2.7 μm+In-line filter 4.6 mm, 0.2 μm filter; column temperature: 30° C.±0.8° C.
detector: Diode Array Detector
CHLOROGENIC ACID: wavelength=325 nm−bandwidth 4.
CYNAROPICRIN: wavelength=212 nm−bandwidth 4.
flow rate: 0.43 ml/min.
injection volume: 5 μl
mobile phase: A=H2O/0.1% HCOOH, B=CH3CN/0.1% HCOOH.

### Elution Conditions:

| min. | A % | B % |
|---|---|---|
| 0 | 92 | 8 |
| 15 | 52 | 48 |

stop time: 15 min.
post time: 5 min.

### Standard Preparation:

Standard: Cynaropicrin—Solubilization solvent: MeOH for HPLC. Working concentration: from 0.00404 to 0.064624 mg/ml. Storage conditions: working solutions are stored at −20° C. and protected from light.

Standard: Chlorogenic acid—solubilization solvent: 50% MeOH for HPLC. Working concentration: 0.02548 to 0.10192 mg/ml. Storage conditions: working solutions are stored at +4° C. and protected from light.

### Chromatoaraphic Conditions (HPLC Method):

### Column: Luna C18 150×4.6 mm 5 μm

column temperature: 30° C.±0.8° C.
detector: Diode Array Detector
CHLOROGENIC ACID: wavelength=325 nm−bandwidth 4. Ref. off
CYNAROPICRIN: wavelength=212 nm−bandwidth 4. Ref.off
flow rate: 0.5 ml/min.
injection volume: 10 μl
mobile phase: A=H2O/0.1% HCOOH, B=CH3CN/0.1% HCOOH

### Elution Conditions:

| min. | A % | B % |
|---|---|---|
| 0 | 92 | 8 |
| 38 | 62 | 38 |
| 45 | 5 | 95 |

stop time: 45 min.
post time: 10 min.

### Calculations:

The percent content of Chlorogenic acid in solid products is calculated with the following formula:

$$\begin{matrix}\% Chlorogenic \\ acid in solid \\ products =\end{matrix} \qquad \frac{AC \times conc . St \times V}{ASt \times p \times 10}$$

### Where:

AC=area of peak related to chlorogenic acid in the sample;
Ast=area of peak related to chlorogenic acid in the standard;
conc.st=conc. in mg/ml of chlorogenic acid in the standard;
V=total volume in ml of the extract;
p=sample weight in grams;
F=dilution factor.

The percent content of cynaropicrin in solid products is calculated with the same formula 15. Determination of total caffeoylquinic acids expressed as chlorogenic acid in Cynara scolymus.

### 10. Determination of Total Caffeoylquinic Acids Expressed as Chlorogenic Acid in Cynara scolymus

Preparation of sample: Accurately weigh 0.30 g±0.015 g of lyophilised extract sample (0.50 g if ground leaves). Add 40 ml of ultrapure H₂O and place under magnetic stirring at the temperature of 95° C.±2° C. Upon reaching the boiling temperature, filter through cotton in a 50 ml centrifuge tube. Add 2 ml of a saturated acetate lead solution to the (still warm) solution.

Cool down, centrifuge and discard the supernatant. Add 5 ml of ultrapure H₂O to the residue and stir the centrifuge tube. Centrifuge again and discard the supernatant. Extract the residue with 70 ml of diluted acetic acid (11.4 ml brought to 100 ml with ultrapure H₂O) and heat to boiling under slow stirring. Filter through cotton the still warm solution and add 2 ml of a (200 ml/1) solution of sulphuric acid. Centrifuge and decant the clear solution in a 100 ml volumetric flask. Add 5 ml of diluted acetic acid to the residue. Centrifuge and decant the clear solution in the same 100 ml flask. At room temperature, bring to a 100 ml volume with diluted acetic acid.

Test solution: take 1 ml of solution. By volumetric flask, bring to 25 ml with methanol and stir.
Reference solution: take 1 ml of acetic acid. By volumetric flask, bring to 25 ml with methanol and stir.

### Spectrophotometric Reading:

Measure test solution absorbance at 325 nm using reference solution as blank. Definition of A1%, 1 cm (as defined in the European Pharmacopoeia Ed 8.0, 2.2.25)=specific absorbance, measured at a specific wavelength, of a reference substance dissolved at the concentration of 10 g/Litre, placed in a 1 cm-long cell.

Assuming the value A1%, 1 cm of the chlorogenic acid at 325 nm to be 485, the percent of caffeoylquinic acids, expressed as chlorogenic acid, is calculated with the formula:

### Calculations:

$$\begin{matrix}\% total caffeoylquinic acids , \\ expressed as chlorogenic \\ acid =\end{matrix} \qquad \frac{A \times Ve \times Vf}{P \times Vp \times A 1 \% , 1 cm}$$

wherein:
A=sample absorbance at 325 nm.
Ve—end volume of the extract.
Vf—end volume of the dilution.
p=sample weight in grams.
Vp—sample volume taken for final dilution.
A1%, 1 cm=485 (A1%, 1 cm of Chlorogenic acid, at a 325 nm wavelength).

### BIBLIOGRAPHY

Aggarwal B. B. et al. Ann. N.Y. Acad. Sci. 1091; 151-69: 2006
Johnston P A e Grandis R G, Mollnterv; 11 (1); 18-26:2011
Niu G. et al. Mol Cancer Res, 6 (7); 1099-105: 2008
Turkson J. Jove R. “STAT proteins: novel molecular targets for cancer drug discovery” Oncogene. 2000 Dec. 27; 19(56):6613-26
Yu. H. et al “STATs in cancer inflammation and immunity: a leading role for STAT3” Nature Reviews Cancer 9, 798-809 (November 2009)

1. An extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form
or wherein
total caffeoylquinic acids represent from 25% to 48% by weight of said fraction in dry form, chlorogenic acid represents from 11% to 21% by weight of said fraction in dry form, and said cynaropicrin represents from 1% to 10% by weight of said fraction in dry form.

2. The extract of Cynara scolymus or fraction of extract of Cynara scolymus or mixture of said extract with one or more of said fractions or mixture of said fractions according to claim 1, wherein
said total caffeoylquinic acids represent from 9% to 15% by weight of said extract or of said fraction or of said mixture in dry form, said chlorogenic acid represents from 3.5% to 5.5% by weight of said extract or of said fraction or of said mixture in dry form, and said cynaropicrin represents from 0.2% to 3% by weight of said extract or of said fraction or of said mixture in dry form
or wherein
said total caffeoylquinic acids represent from 25% to 35% by weight of said fraction in dry form, said chlorogenic acid represents from 11% to 15% by weight of said fraction in dry form, and said cynaropicrin represents from 1% to 8% by weight of said fraction in dry form.

3. The extract of Cynara scolymus or fraction of extract of Cynara scolymus or mixture of said extract with one or more of said fractions or mixture of said fractions according to claim 1, wherein said extract, said fraction, or said mixture is in a dry, lyophilised or fluid form and is obtained from Cynara leaves, flower-heads, or mixtures thereof.

4. The extract of Cynara scolymus or fraction of extract of Cynara scolymus or mixture of said extract with one or more of said fractions or mixture of said fractions according to claim 1, in association with one or more anti-tumour compounds.

5. The extract of Cynara scolymus or fraction of extract of Cynara scolymus or mixture of said extract with one or more of said fractions or mixture of said fractions according to claim 4, wherein said association is carried out by concomitant or sequential administration of said extract or of said fraction, or of said mixture, with said one or more anti-tumour compounds.

6. The extract of Cynara scolymus or fraction of extract of Cynara scolymus or mixture of said extract with one or more of said fractions or mixture of said fractions according to claim 4, wherein said one or more anti-tumour compounds are selected from the group consisting of cisplatinum, doxorubicin, pemetrexed, methotrexate, vinorelbine, gemcitabine, and taxol.

7. A composition comprising as sole active pharmaceutical ingredient, an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form;
or wherein
total caffeoylquinic acids represent from 25% to 48% by weight of said fraction in dry form, chlorogenic acid represents from 11% to 21% by weight of said fraction in dry form, and said cynaropicrin represents from 1% to 10% by weight of said fraction in dry form;
and a carrier and/or diluent and/or excipient.

8. The composition according to claim 7, wherein
said total caffeoylquinic acids represent from 9% to 15% by weight of said extract or of said fraction or of said mixture in dry form, said chlorogenic acid represents from 3.5% to 5.5% by weight of said extract or of said fraction or of said mixture in dry form, and said cynaropicrin represents from 0.2% to 3% by weight of said extract or of said fraction or of said mixture in dry form;
or wherein
said total caffeoylquinic acids represent from 25% to 35% by weight of said fraction in dry form, said chlorogenic acid represents from 11% to 15% by weight of said fraction in dry form, and said cynaropicrin represents from 1% to 8% by weight of said fraction in dry form.

9. The composition according to claim 7, further comprising, beside said active pharmaceutical ingredient, one or more anti-tumour compounds.

10. The composition according to claim 7, wherein said one or more anti-tumour compounds are selected from the group consisting of cisplatinum, doxorubicin, pemetrexed, methotrexate, vinorelbine, gemcitabine, and taxol.

11. A kit for concomitant or sequential administration of an extract of Cynara scolymus or a fraction of extract of Cynara scolymus or a mixture of said extract with one or more of said fractions or a mixture of said fractions and one or more anti-tumour compounds, comprising:
one or more aliquots of an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein total caffeoylquinic acids represent from 8% to 16% by weight of said extract or of said fraction or of said mixture in dry form, chlorogenic acid represents from 3.5% to 8% by weight of said extract or of said fraction or of said mixture in dry form, and said cynaropicrin represents from 0.2% to 4% by weight of said extract or of said fraction or of said mixture in dry form, and
one or more aliquots of one or more anti-tumour compounds.

12. The kit according to claim 11, wherein said total caffeoylquinic acids represent from 9% to 15% by weight of said extract or of said fraction or of said mixture in dry form, said chlorogenic acid represents from 3.5% to 5.5% by weight of said extract or of said fraction or of said mixture in dry form, and said cynaropicrin represents from 0.2% to 3% by weight of said extract or of said fraction or of said mixture in dry form.

13. The kit according to claim 11, wherein said one or more anti-tumour compounds are selected from the group consisting of cisplatinum, doxorubicin, pemetrexed, methotrexate, vinorelbine, gemcitabine, and taxol.

14. A method of using the extract of Cynara scolymus or fraction of extract of Cynara scolymus or mixture of said extract with one or more of said fractions or mixture of said fractions according to claim 1 for prevention and/or treatment of malignant pleural mesothelioma.

15. A method of using the composition according to claim 7 for prevention and/or treatment of malignant pleural mesothelioma.

16. A method of using the kit according to claim 11 for prevention and/or treatment of malignant pleural mesothelioma.
